SEQUENCE LISTING (26 sequences)

SEQ ID NO: 1 - DNA, 24 bp, Artificial
  Primer
ctgcattaaaacattcgaaaccaa

SEQ ID NO: 2 - DNA, 23 bp, Artificial
  Primer
gcagcagattgtgggacttctca

SEQ ID NO: 3 - DNA, 19 bp, Artificial
  Primer
agcctgatcattaccaggt

SEQ ID NO: 4 - DNA, 22 bp, Artificial
  Primer
gtttctattggggccatcttgc

SEQ ID NO: 5 - DNA, 25 bp, Artificial
  Primer
tctctctctccctctctctttcgag

SEQ ID NO: 6 - DNA, 28 bp, Artificial
  Primer
gtttgccatcttcttacctctgttggtc

SEQ ID NO: 7 - DNA, 23 bp, Artificial
  Primer
caggatattatgtgatggaatcc

SEQ ID NO: 8 - DNA, 27 bp, Artificial
  Primer
gatctcttctctctctctctttctccc

SEQ ID NO: 9 - DNA, 23 bp, Artificial
  Primer
tatggtgaccttaagcagccatg

SEQ ID NO: 10 - DNA, 28 bp, Artificial
  Primer
gtgtcttagctgagagatcacgcactgc

SEQ ID NO: 11 - DNA, 20 bp, Artificial
  Primer
ggtagcagagcaagactgtc

SEQ ID NO: 12 - DNA, 24 bp, Artificial
  Primer
gtttcaccttcatcctaaggcagc

SEQ ID NO: 13 - DNA, 20 bp, Artificial
  Primer
actactccaaggttccagcc

SEQ ID NO: 14 - DNA, 22 bp, Artificial
  Primer
actgttatctctcttgtggtag

SEQ ID NO: 15 - DNA, 23 bp, Artificial
  Primer
atcaacaggatcaatggatgcat

SEQ ID NO: 16 - DNA, 22 bp, Artificial
  Primer
gggcttttagacctggactgag

SEQ ID NO: 17 - DNA, 19 bp, Artificial
  Primer
ccaacagagcaagactgtc

SEQ ID NO: 18 - DNA, 25 bp, Artificial
  Primer
gtttgaaacagttaaatggccaaag

SEQ ID NO: 19 - DNA, 20 bp, Artificial
  Primer
atagacagacagacggactg

SEQ ID NO: 20 - DNA, 23 bp, Artificial
  Primer
gtttgggagtggagattacccct

SEQ ID NO: 21 - DNA, 20 bp, Artificial
  Primer
aaccgtagcctggcgagact

SEQ ID NO: 22 - DNA, 26 bp, Artificial
  Primer
gtttccttttaagctgcaacaatttc

SEQ ID NO: 23 - DNA, 23 bp, Artificial
  Primer
gttccacataatgaagcacaatc

SEQ ID NO: 24 - DNA, 23 bp, Artificial
  Primer
gtttaatcgcctgagtcctggga

SEQ ID NO: 25 - DNA, 20 bp, Artificial
  Primer
aatctgggcgacaagagtga

SEQ ID NO: 26 - DNA, 20 bp, Artificial
  Primer
acatctcccctaccgctata

(19) United States
(12) Patent Application Publication
(10) Pub. No.: US 2010/0099082 A1
(43) Pub. Date: Apr. 22, 2010

(54) Allele Detection

(75) Inventors: Thomas Lion, Vienna (AT); Peter Bader, Frankfurt am Main (DE); Helene Cave, Paris (FR); Mark Lawler, Dublin (IE); Andrea Biondi, Monza (IT); Anna Serra, Orbassano-Torino (IT); Jacques J. M. Van Dongen, Rotterdam (NL); Marcel G.J. Tilanus, Maastricht (NL); Marcos Gonzalez Diaz, Salamanca (ES); Gisela Barbany, Uppsala (SE); Eddy Roosnek, Geneve (CH); Colin G. Steward, Bristol (GB)

Correspondence Address: FULBRIGHT & JAWORSKI L.L.P., 600 CONGRESS AVE., SUITE 2400, AUSTIN, TX, 78701, US

(73) Assignee: ST. ANNA KINDERKREBSFORSCHUNG, Vienna (AT)

(21) Appl. No.: 12/303,075

(22) PCT Filed: Jun. 1, 2007
(86) PCT No.: PCT/AT2007/000266

§ 371 Date: Dec. 14, 2009

(30) Foreign Application Priority Data
Jun. 1, 2006 (AT) A 948/2006

(51) Int. Cl. C12Q 1/68; C07H 21/00; G01N 27/26

(52) U.S. Cl. 435/6; 536/24.33; 204/450; 204/451; 204/456

(57) ABSTRACT

Method for simultaneously determining alleles present in a set of loci from at least one nucleic acid sample comprising the steps: a) providing said at least one sample, b) subjecting said sample to a nucleic acid amplification reaction using a primer pair simultaneously primer pairs specific and optimized for each of the loci of a set of at least three loci selected from the group consisting of D2S1360, D7S1517, D8S1132, D9S1118, D10S2325, D11S554, D12S1064, D12S391, D17S1290, D19S253, MYCLl, P450CYP19 and SE-33, and c) evaluating the length and optionally the relative quantity of amplification products obtained from step b) or from the analysis of one or two of the above loci to determine and/or optionally quantify the alleles present at each of the loci analyzed in the set within said sample.

The present invention relates to a method for simultaneously determining and quantifying microsatellite alleles.

In recent years, the discovery and development of polymorphic short tandem repeats (STRs) as genetic markers has stimulated progress in the identification and characterization of diseases caused by genetic defects and in forensic DNA typing.

Short tandem repeats (STRs), also called microsatellites, are tandemly repeated units of DNA distributed throughout the human genome (see e.g. Hohoff et al. (1999) Mol. Biotech. 13:123-136). The repeating units are typically of two to seven base pairs. In certain instances, the size of an STR may be hundreds of base pairs, depending on the number of repeating units. The number of repeating units varies among individuals. The polymorphic nature of STRs allows them to be used in various methods, including genetic linkage studies (e.g. paternity testing), forensic DNA typing and clinical diagnostics. Therefore, STR loci are extremely useful markers for human identification, paternity testing and genetic mapping. STR loci may be amplified via a nucleic acid amplification technique, like polymerase chain reaction (PCR) by employing specific primer sequences identified in the regions flanking the tandem repeat.

To minimize labor, materials and analysis time, it is desirable to analyze multiple loci of one or more samples simultaneously by a multiplex approach. Such “multiplex” amplifications have been described extensively in the literature (e.g. Thiede C, et al. Bone Marrow Transplant (1999) 23: 1055-1060; Pindolia K, et al. Bone Marrow Transplant (1999) 24: 1235-1241; Nollet F, et al. Bone Marrow Transplant (2001) 28: 511-518; Sanchez J J, et al. Electrophoresis (2006), PMID:16586411).

WO 92/021693 relates to polymorphic marker which may be used particularly in forensic medicine and in gene mapping.

WO 97/39138 relates to methods for the simultaneous amplification of gene segments of various loci. The method described in said document is particularly suited for forensic medicine, paternity testing and gene mapping.

AU 717 638 discloses a method to establish the genetic profile of an individual by amplifying 8 highly polymorphic short tandem repeat loci.

Dubovsky et al. (Leukemia 13 (12) (1999): 2060-2069) describes a method for monitoring chimerism by using polymerase chain reaction. The amplification of the markers in Dubovsky et al. was performed separately under identical conditions.

Especially in clinical diagnostics the identification of alleles is becoming more and more important. In particular, the transplantation of hematopoietic stem cells from related or unrelated donors is becoming an increasingly important approach to treat different malignant and non-malignant disorders. There is thus growing demand for clinical diagnostic methodologies permitting the surveillance of donor- and recipient-derived hemopoiesis (=chimerism) during the post-transplant period. The techniques currently used are very heterogeneous, rendering uniform evaluation and comparison of diagnostic results between laboratories difficult. Therefore, the development of a standardized diagnostic methodology for the detection and monitoring of chimerism, progress of treatment and risk evaluation in patients undergoing allogeneic stem cell transplantation (SCT) is of major importance. However, such methods should also be usable in other areas where the determination of alleles is required.

The present invention relates to a method for simultaneously determining and quantifying alleles present in a set of loci from at least one nucleic acid sample comprising the steps:

a) providing said at least one sample,

b) subjecting said sample to a nucleic acid amplification reaction using simultaneously primer pairs specific for each of the loci of a set of at least three loci selected from the group consisting of D2S1360, D7S1517, D8S1132, D9S1118, D10S2325, D11S554, D12S1064, D12S391, D17S1290, D19S253, MYCL1, P450CYP19 and SE-33, and

c) evaluating the length of amplification products obtained from step b) to determine the alleles present at each of the loci analyzed in the set within said sample.

Optionally, in post-transplantation specimens, for instance, the relative quantity of patient- and recipient-derived cells in the sample may be determined by evaluating the amount of individual amplified allele fragments. The quantification of the amplification products allows to determine the relative amount of cells of varying origins, provided that the cells comprise different alleles.

The method of the present invention contemplates selecting an appropriate set of loci, primers, and amplification protocols to generate amplified alleles from individual or multiple co-amplified loci which preferably do not overlap in size or, more preferably, which are labelled in a way permitting the differentiation between the alleles from different loci overlapping in size. In addition, this method contemplates the selection of short tandem repeat loci which are compatible for use with a single amplification protocol. The specific combinations of loci described herein are unique in this application. Combinations of loci may be rejected for either of the above two reasons, or because, in combination, one or more of the loci do not provide adequate product yield, or fragments which do not represent authentic alleles are produced in this reaction.

Successful combinations in addition to those disclosed herein can be generated by trial and error of locus combinations, by selection of primer pair sequences and by adjustment of primer concentrations to identify an equilibrium in which all included loci may be amplified. Once the method and materials of this invention are disclosed, various methods of selecting loci, primer pairs and amplification techniques for use in the method and kit of this invention are likely to be suggested to one skilled in the art. All such methods are intended to be within the scope of the present invention.

Of particular importance in the practice of the method according to this invention is the size range of amplified alleles produced from the individual loci which are co-amplified in the multiplex amplification reaction step. The amplified fragments of the present invention are preferably smaller than 1000, more preferred smaller than 500, in particular smaller than 400, bases.

Any one of a number of different techniques can be used to select a set of loci for use in the present invention. One preferred technique for developing useful sets of loci for use in this method of analysis is described below. A strategy for selecting an appropriate combination of loci is to initially select two, preferably three, STR loci. Once a multiplex containing two, preferably three, STR loci is developed, it may be used as a core to create multiplexes containing at least three, preferably more than three, loci. New combinations of at least three loci can, thus, be created which include the first two, preferably three, loci.

It is contemplated that core sets of loci can be used to generate other appropriate derivative sets of STR loci for multiplex analysis using the method of this invention. Regardless of which method is used to select the loci analyzed using the method of the present invention, all the loci selected for multiplex analysis share the following characteristics: (1) they produce sufficient amplification product to allow evaluation, (2) they generate few, if any, artifacts due to the addition (or lack of addition) of a base to the amplified alleles during the multiplex amplification step, (3) they generate few, if any, artifacts due to premature termination of amplification reactions by a polymerase, and (4) they produce little or no bands of smaller molecular weight from consecutive single base deletions below a given authentic amplified allele.

According to the present invention the loci to be amplified may be selected from a set consisting of at least three, preferably at least four, more preferably at least five, most preferably at least ten, STR markers. However, the set of loci selected for co-amplification and analysis according to the invention preferably may further comprise at least one locus in addition to the at least three STR loci. Hence, it is of course possible to combine at least three markers of the list of loci according to the method according to the present invention with other markers known in the art (see e.g. Acquaviva C, at al. Leukemia (2003) 17:241-246; Hancock J P, et al. Leukemia (2003) 17:247-251; Kreyenberg H, et al. Leukemia (2003) 17:237-240).

The targeted loci can be co-amplified in the multiplex amplification step of the present method. Any one of a number of different amplification methods can be used to amplify the loci, including, but not limited to, polymerase chain reaction (PCR) (Saiki, R. K., et al. (1985), Science 230: 1350-1354), transcription based amplification (Kwoh, D. Y., and Kwoh, T. J. (1990), American Biotechnology Laboratory, October, 1990) and strand displacement amplification (SDA) (Walker, G. T., et al. (1992) Proc. Natl. Acad. Sci., U.S.A. 89: 392-396). Preferably, the nucleic acid sample is subjected to a PCR amplification using primer pairs specific to each locus in the set.

As used herein, “allele” is intended to be a genetic variation associated with a segment of DNA, i.e., one of two or more alternate forms of a DNA sequence occupying the same locus.

The term “locus” (or genetic locus) refers to a specific position on a chromosome. Alleles of a locus are located at identical sites on homologous chromosomes.

As used herein, “simultaneous determining” means that the alleles in set of at least three loci are determined using at least three primer pairs specific for said loci in the same amplification reaction (“simultaneously”). Such reactions are also called “multiplex” reactions (e.g. if the nucleic acid amplification reaction is a polymerase chain reaction (PCR) “multiplex PCR”).

Detailed information about the microsatellite markers as used in the present invention is available at the NCBI Entrez UniSTS and other web sites (www.ncbi.nlm.nih.qov/entrez/; www.cstl.nist.qov/biotech/strbase/; www.qdb.orq/; www.ensembl.orq/index.html; qai.nci.nih.qov/CHLC/; genome.ucsc.edu). On said web sites suitable primer pairs can also be found.

According to a preferred embodiment of the present invention the set of at least three loci consists of D11S554, D7S1517, D8S1132, D9S1118 and MYCL1; D2S1360, D10S2325, D12S391 and P450CYP19; D11S554, D7S1517 and D8S1132; D11S554, D8S1132 and D9S1118; D11S554, D7S1517 and MYCL1; D11S554, D7S1517, D9S1118 and MYCL1; D11s554, D8s1132 and MYCL1; D11s554, D7s1517 and D9s1118; D1s554, MYCL1 and D9s1118; D11S554, D7S1517, D9S1118 and MYCL1; D11s554, D8s1132, D7S1517 and MYCL1; D11S554, D8S1132, MYCL1 and D9S1118; D11s554, D8S1132, D7s1517 and D9S1118; D10s2325, P450CYP19 and D2S1360; D10S2325, D12S391 and P450CYP19; D10S2325, D12S391 and D2S1360; D10S2325, D12S391 and D2S1360.

Particularly preferred combinations of loci to be detected in the course of the method according to the present invention are outlined above. However, it is evident that in practice every combination of at least three loci disclosed herein may be combined.

According to another preferred embodiment of the present invention said at least one nucleic acid sample is obtained from a transplantation recipient prior and after subjecting said recipient to transplantation from a donor.

The method according to the present invention may be suitably employed for analyzing the success, status and/or progress of a transplantation of, e.g., bone marrow, from a donor to a recipient. In the course of the treatment cells (e.g. leukocytes) of the recipient are preferably substituted by cells of the donor leading in some stages of the treatment to a chimeric state in the recipient.

Chimerism analysis has become a routine method to document engraftment and also for detection of residual disease. Nucleic acid amplification-based procedures using STR analysis, particularly in multiplex assays, are frequently used. However, these assays have been optimized for forensic purposes and do not necessarily fulfil all needs for chimerism analysis.

Microsatellite (STR) markers, selected on the basis of their excellent performance in chimerism analysis, have been carefully evaluated and optimized for quantitative chimerism testing under standardized experimental conditions. The 13 markers (loci) disclosed herein optimally meet the specific requirements of quantitative chimerism analysis. The ability of the marker panel to provide informative markers for the monitoring of chimerism was shown to be superior to commercial microsatellite panels for forensic purposes. In addition to the outstanding informativeness of the marker panel, the standardized chimerism assay according to the present invention permits sensitive detection of residual cells of any origin at a level ranging between 0.8-1.6% in the great majority of instances. Moreover, the method of the present invention facilitates accurate and reproducible quantification of donor and recipient hematopoietic cells.

The requirements for the eligibility of microsatellite markers for clinical testing of chimerism are far more stringent than those for forensic analysis. The allelic constellations eligible for application in chimerism testing require not merely different allelic patterns of donor and recipient, but, as indicated above, a number of additional features relevant for quantitative analysis of allele ratios (see Lion T, Leukemia (2003) 17:252-254).

Multiplex microsatellite kits known in the art permit simultaneous amplification of several STR loci, but the markers included generally provide a level of informativeness for the purpose of chimerism analysis inferior to the marker panel according to the present invention. Most commercial kits, e.g. the Powerplex (Promega) of the Identifiler (ABI), have been developed for forensic analysis and therefore do not provide a comparable number of microsatellite markers eligible for chimerism testing. By contrast, the marker panel of the present invention has been extensively tested for the frequencies of individual alleles and was shown to provide a minimum of two informative markers in virtually any donor-recipient constellation. The multiplex reactions of the marker panel therefore permit the selection of several markers optimally suited for the follow-up of chimerism during the post-transplant period. Upon identification of one or more informative markers in a given donor/recipient constellation, the marker panel of the present invention provides optimized primers and reaction conditions for quantitative monitoring of chimerism in singleplex reactions. Alternatively, amplification primers of two (or three) selected informative markers can be combined in a duplex (or triplex) reaction and quantitative analysis of chimerism can be performed by calculating the mean of the readouts for each individual marker included in the reaction. The possibility of multiplexing a small number of markers selected on the basis of their informativeness in a particular donor/recipient situation combines the advantages of obtaining an extended set of data for quantitative analysis in a single reaction while maintaining a high level of sensitivity.

Existing commercial multiplex tests used for chimerism testing (Biotype, Serac) do not provide optimized primers and protocols for singleplex PCR reactions facilitating precise assessment of chimerism in patient specimens. With these kits, quantitative chimerism testing can only be performed by employing the entire multiplex reaction, which does not provide the same level of sensitivity as singleplex (or limited oligoplex) reactions.

The marker panel has been established and evaluated in a large series of experiments. The accurate selection of suitable loci and the optimized primer composition of the multiplex and singleplex PCR reactions according to the present invention provide a unique system for reliable and accurate investigation of chimerism in the routine clinical setting.

The term “informativeness”/“informative marker” in the context of the intended use of the present invention describes the probability of a given microsatellite panel (marker panel, loci) to provide one or more markers eligible in particular for quantitative chimerism analysis.

In contrast to the use of microsatellite markers for forensic applications (e.g. paternity testing, person identification), which are qualitative in nature and where any differences in the allelic constellations can be regarded as informative, the application for chimerism analysis has far more stringent requirements. Chimerism analysis is a quantitative technique and is therefore influenced by additional criteria pertaining to the type of allelic constellations. These include primarily the extent of stutter peak formation and the distances (i.e. differences in size) between individual alleles. Microsatellite markers providing high informativeness for chimerism analysis must have a high probability of yielding alleles located between two to four repeat units from each other. Hence, they must be located outside each others stutter areas (i.e. more than one repeat unit apart) and the distance should not be too large, to prevent the effect of unequal amplification efficiency on the quantitative analysis. The microsatellite panel in the current invention has been judiciously selected and evaluated for the frequency of alleles and allelic constellations meeting the requirements of informativeness for chimerism testing.

The above implies that microsatellite panels designed for the use in forensic medicine are not likely to meet the criteria of applicability for quantitative chimerism analysis in a fashion comparable to the microsatellite panel of the present invention which has specifically been designed for the latter application.

According to a preferred embodiment of the present invention at least one further nucleic acid sample is obtained from said donor.

In order to determine the presence of cells of the donor in the recipient after transplantation, the alleles of the donor may preferably be analyzed too. This allows to unambiguously attribute alleles found in the recipient to the donor or, if the transplantation did not succeed and the recipient still produces own cells, to the recipient himself.

According to a preferred embodiment of the present invention said recipient is transplanted with donor tissue, preferably with bone marrow or enriched hematopoietic stem cells.

The method according to the present invention is especially suited to monitor the transplantation of a donor tissue, in particular bone marrow or hematopoietic stem cells, to a recipient.

The determined length of the fragments obtained by step b) of the method according to the present invention of the at least one sample of the recipient after transplantation is preferably compared to the at least one sample of the recipient prior to transplantation or to the at least one sample of the donor.

The determination of the length of the fragments obtained by the nucleic acid amplification allows creating an allelic profile of the analyzed sample. This profile may then be used for comparison to the other samples.

According to another preferred embodiment of the present invention said sample is a blood sample or a bone marrow sample.

Of course it is also possible to use other nucleic acid sources like tissues and body fluids, selected from the group consisting of semen, vaginal cells, hair, bone, buccal samples, amniotic fluid containing placental cells or fetal cells, chorionic villi and mixtures of any of the tissues listed above. However, if the recipient was subjected to a bone marrow transplantation, it is particularly preferred to provide a blood sample, which can be used in a method according to the present invention.

The sample used in a method of the present invention preferably comprises nucleated cells, in particular leukocytes and/or stem cells.

If the sample is a blood or bone marrow sample the preferred nucleic acid source are leukocytes. Since leukocytes are formed in the bone marrow the nucleic acid obtained from leukocytes is particularly suited for determining alleles in bone marrow transplanted recipients. The leukocytes to be analyzed may be purified or used directly in their natural matrix (e.g. blood). Leukocytes may be employed in the method according to the present invention directly without isolating their nucleic acid (e.g. DNA) or their nucleic acid is extruded, isolated and analyzed. In an especially preferred embodiment of the present invention the nucleic acid is isolated and optionally enriched by methods known in the art.

According to a preferred embodiment of the present invention the leukocytes are granulocytes, monocytes, lymphocytes, preferably NK cells, B cells or T cells, in particular T helper cells, T suppressor cells, in particular cells expressing CD3, CD4, CD8, CD14, CD15, CD19, CD34, CD38, CD45 and/or CD56.

The forward and/or the reverse primers are preferably labelled with a fluorescent marker.

In order to detect the amplification products obtained by the method according to the present invention the forward and/or reverse primers may be conjugated with a detectable label. Said detectable label is preferably a fluorescent label (marker), but it is of course also possible to use alternative labels known in the art.

According to a preferred embodiment of the present invention the fluorescent marker is selected from the group consisting of Alexa 350, Alexa 430, AMCA, FL, R6G, TMR, TRX, Cascade Blue, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine and Texas Red.

The method according to the present invention is most preferably practised using fluorescent detection as the detection step. In this preferred method of detection, one or both of each pair of primers used in the multiplex or singleplex amplification reaction has a fluorescent label attached or conjugated thereto, and as a result, the amplified alleles produced from the amplification reaction are fluorescently labelled. In this most preferred embodiment of the invention, the amplified alleles are subsequently separated, e.g., by capillary electrophoresis and the separated alleles visualized and analyzed using a fluorescent image analyzer.

The use of different fluorescent labels is especially advantageous when the amplification products obtained exhibit similar length, because in such a case it is only possible to distinguish different amplified loci by their varying fluorescent features.

The primer pairs have, preferably primer sequences SEQ ID No. 1 and SEQ ID No. 2 if the locus to be amplified is D2S1360; SEQ ID No. 3 and SEQ ID No. 4 if the locus to be amplified is D7S1517; SEQ ID No. 5 and SEQ ID No. 6 if the locus to be amplified is D8S1132; SEQ ID No. 7 and SEQ ID No. 8 if the locus to be amplified is D9S1118; SEQ ID No. 9 and SEQ ID No. 10 if the locus to be amplified is D10S2325; SEQ ID No. 11 and SEQ ID No. 12 if the locus to be amplified is D11S554; SEQ ID No. 13 and SEQ ID No. 14 if the locus to be amplified is D12S1064; SEQ ID No. 15 and SEQ ID No. 16 if the locus to be amplified is D12S391; SEQ ID No. 17 and SEQ ID No. 18 if the locus to be amplified is D17S1290; SEQ ID No. 19 and SEQ ID No. 20 if the locus to be amplified is D19S253; SEQ ID No. 21 and SEQ ID No. 22 if the locus to be amplified is MYCL1; SEQ ID No. 23 and SEQ ID No. 24 if the locus to be amplified is P450CYP19 and SEQ ID No. 25 and SEQ ID No. 26 if the locus to be amplified is SE-33. Of course it is also possible to use primers known in the art alone or in combination with the primers according to the present invention.

Primers and primer pairs are preferably developed and selected for use in the multiplex systems of the invention by employing a re-iterative process of selecting primer sequences, mixing the primers for co-amplification of the selected loci, co-amplifying the loci, then separating and detecting the amplified products. Initially, this process often produces the amplified alleles in an imbalanced fashion (i.e., higher product yield for some loci than for others) and may also generate amplification products, which do not represent the alleles themselves. These extra fragments may result from any number of causes described above.

To eliminate such extra fragments from the multiplex systems, individual primers from the total set are used with primers from the same or other loci to identify which primers contribute to the amplification of the extra fragments. Once two primers which generate one or more of the fragments are identified, one or both contributors are modified and retested, either in a pair alone or in the multiplex system (or a subset of the multiplex system). This process is repeated until evaluation of the products yields amplified alleles with no or an acceptable level of extra fragments in the multiplex system.

The determination of primer concentration may be performed either before or after selection of the final primer sequences, but is preferably performed after that selection. Generally, increasing primer concentration for any particular locus increases the amount of product generated for that locus. However, this is also a re-iterative process because increasing yield for one locus may decrease it for one or more other loci. Furthermore, primers may interact, directly affecting yield of the other loci. Linear increases in primer concentration do not necessarily produce linear increases in product yield for the corresponding locus.

Locus to locus balance is also affected by a number of parameters of the amplification protocol such as the amount of template used, the number of cycles of amplification, the annealing temperature of the thermal cycling protocol, and the inclusion or exclusion of an extra extension step at the end of the cycling process. Absolutely even balance across all alleles and loci is generally not achieved.

According to a preferred embodiment of the present invention the nucleic acid of the nucleic acid sample is isolated from a nucleic acid comprising source.

Nucleic acid comprising samples of a nucleic acid source can be prepared for use in the method according to the present invention using any method of nucleic acid preparation which is compatible with the amplification of nucleic acids, in particular of DNA. Many such methods are known to those skilled in the art. Examples include, but are not limited to DNA purification by phenol extraction (Sambrook, J., et al. (2001) Molecular Cloning: A Laboratory Manual, Third Edition, Cold Spring Harbor Laboratory Press, Cold Spring Harbor, N.Y.), and partial purification by salt precipitation (Miller, S. et al. (1988) Nucl. Acids Res. 16:1215) or chelex (Walsh et al., (1991) BioTechniques 10:506-513, Comey, et al., (1994) Forensic Sci. 39:1 254) and the release of unpurified material using untreated blood (Burckhardt, J. (1994) PCR Methods and Applications 3:239-243, McCabe, Edward R. B., (1991) PCR Methods and Applications 1:99-106, Nordvag, Bjorn-Yngvar (1992) BioTechniques 12:4 pp. 490-492). However, it is of course also possible to use a sample where nucleic acid is not extracted nor (highly) purified from the nucleic acid comprising source (e.g. blood).

According to a preferred embodiment of the present invention the lengths of the amplification products are evaluated by electrophoresis, preferably by gel or capillary electrophoresis.

Once a set of amplified alleles is produced from the multiplex amplification step of the present method, the amplified alleles are evaluated. The evaluation step of this method can be accomplished by any one of a number of different means, the most preferred of which are described below. The length of the amplification products obtained by the method according to the present invention may be determined by all suitable methods known in the art. However, a preferred method includes electrophoresis, in particular gel or capillary electrophoresis.

Electrophoresis is preferably used to separate the products of the multiplex amplification reaction, more preferably capillary electrophoresis (see, e.g., Buel, Eric et al. (1998), Journal of Forensic Sciences; 43: 164-170) or denaturing polyacrylamide gel electrophoresis (see, e.g., Sambrook, J. et al. (2001) In Molecular Cloning-A Laboratory Manual, 3rd edition, Cold Spring Harbor Laboratory Press). Gel preparation and electrophoresis procedures and conditions suitable for use in the evaluating step of the method of this invention are known to the person skilled in the art. Separation of amplified DNA fragments in a denaturing polyacrylamide gel and in capillary electrophoresis occurs primarily based on fragment size.

Once the amplified alleles are separated, the alleles and any other DNA in the gel or capillary (e.g., DNA size markers or an allelic ladder) can then be visualized and analyzed. Visualization of the DNA in the gel can be accomplished using any one of a number of prior art techniques, including silver staining or reporters such as radioisotopes, fluorescent labels, chemiluminescent labels and enzymes in combination with detectable substrates. However, the preferred method for detection of multiplexes containing at least three loci is fluorescence (see, e.g., Schumm, J. W. et al. in Proceedings from the Eighth International Symposium on Human Identification, (pub. 1998 by Promega Corporation), pp. 78-84; Buel, Eric et al. (1998), supra.), wherein primers for each locus in the multiplexing reaction is followed by detection of the labelled products employing a fluorometric detector.

The fragments representing the alleles present in the nucleic acid sample are preferably determined by comparison to a size standard such as a DNA marker or a locus-specific allelic ladder to determine the alleles present at each locus within the sample. The most preferred size marker for evaluation of a multiplex amplification containing two or more polymorphic STR loci consists of a combination of allelic ladders for each of the loci being evaluated. See, e.g., Puers, Christoph et al., (1993) Am J. Hum Genet. 53:953-958, Puers, Christoph, et al. (1994) Genomics 23:260-264. See also, U.S. Pat. No. 5,599,666; U.S. Pat. No. 5,674,686; and U.S. Pat. No. 5,783,406 for descriptions of allelic ladders suitable for use in the detection of STR loci, and methods of ladder construction disclosed therein.

Following the construction of allelic ladders for individual loci, these may be mixed and loaded for gel electrophoresis at the same time as the loading of amplified samples occurs. Each allelic ladder co-migrates with alleles in the sample from the corresponding locus.

The products of the multiplex reactions of the present invention can be evaluated using an internal lane standard, a specialized type of size marker configured to run in the same lane of a polyacrylamide gel or in the same capillary. The internal lane standard preferably consists of a series of fragments of known length. The internal lane standard is more preferably labelled with a fluorescent dye which is distinguishable from other dyes in the amplification reaction.

Following construction of the internal lane standard, this standard can also be mixed with amplified sample or allelic ladders and loaded for electrophoresis for comparison of migration in different lanes of gel electrophoresis or different capillaries of capillary electrophoresis. Variation in the migration of the internal lane standard indicates variation in the performance of the separation medium. Quantitation of this difference and correlation with the allelic ladders allows correction in the size determination of alleles in unknown samples.

It is also possible to determine the alleles present in a sample or the allelic profile by using microarrays (e.g. DNA microarrays). The hybridization of the amplified products may be performed, for instance, on micro or nano particles (see, e.g., Heller M J, Annu Rev Biomed Eng. 4 (2002):129-153).

The loci D11S554, D7S1517, D8S1132, D9S1118 and MYCL1 are preferably simultaneously amplified in a first amplification reaction and the loci D2S1360, D10S2325, D12S391 and P450CYP19 are preferably simultaneously amplified in a second amplification reaction.

In order to achieve even better results it is possible to perform more than one (preferably two, more preferably three, most preferably five) amplification reactions wherein in each of said reactions at least three loci selected from the group according to the present invention are amplified.

Another aspect of the present invention relates to a kit for determining alleles present in a set of loci from at least one nucleic acid sample or for determining fragment lengths of alleles or for detecting chimerism in a transplant recipient comprising primer pairs each specific for at least three loci selected from the group consisting of D2S1360, D7S1517, D8S1132, D9S1118, D10S2325, D11S554, D12S1064, D12S391, D17S1290, D19S253, MYCL1, P450CYP19 and SE-33.

The kit according to the present invention which comprises primer pairs can be suitably employed in any method which requires the determination of alleles in a sample. For instance, said kit may be used for detecting and quantifying chimerism in an individual, for paternity testing, for forensic analysis etc. The kit may further comprise allelic ladders (see e.g. U.S. Pat. No. 5,599,666), allelic ladders directed to each of the specified loci, positive controls, buffers, etc.

The forward and/or the reverse primers are preferably labelled, preferably labelled with a fluorescent marker.

According to a preferred embodiment of the present invention the fluorescent marker is selected from the group consisting of Alexa 350, Alexa 430, FL, R6G, TMR, TRX, Cascade Blue, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine and Texas Red.

The primer pairs have preferably primer sequences SEQ ID No. 1 and SEQ ID No. 2 if the locus to be amplified is D2S1360; SEQ ID No. 3 and SEQ ID No. 4 if the locus to be amplified is D7S1517; SEQ ID No. 5 and SEQ ID No. 6 if the locus to be amplified is D8S1132; SEQ ID No. 7 and SEQ ID No. 8 if the locus to be amplified is D9S1118; SEQ ID No. 9 and SEQ ID No. 10 if the locus to be amplified is D10S2325; SEQ ID No. 11 and SEQ ID No. 12 if the locus to be amplified is D11S554; SEQ ID No. 13 and SEQ ID No. 14 if the locus to be amplified is D12S1064; SEQ ID No. 15 and SEQ ID No. 16 if the locus to be amplified is D12S391; SEQ ID No. 17 and SEQ ID No. 18 if the locus to be amplified is D17S1290; SEQ ID No. 19 and SEQ ID No. 20 if the locus to be amplified is D19S253; SEQ ID No. 21 and SEQ ID No. 22 if the locus to be amplified is MYCL1; SEQ ID No. 23 and SEQ ID No. 24 if the locus to be amplified is P450CYP19 and SEQ ID No. 25 and SEQ ID No. 26 if the locus to be amplified is SE-33.

Another aspect of the present invention relates to the use of a method according to the present invention for the detection of chimerism in a transplant recipient.

Lawler et al. (Blood 1991; 77:2504-2514) were the first to report the use of PCR for the amplification of highly polymorphic STR sequences in the field of chimerism analysis, and numerous assays based on variable number of tandem repeat (VNTR) or STR markers have been reported since then. Especially multiplex amplification of STR markers with fluorescence detection was described, since such approach allows the rapid identification of informative markers and also enables the calculation of mean values, which increases the accuracy and reproducibility of the results. Most known multiplex STR systems are designed for forensic purposes and are, however, frequently used for chimerism analysis. Although forensic analysis requires a high degree of informativeness and standardization in respect to their purpose to unambiguously identify individuals, the use of said multiplex STR systems in determining chimerism is not necessarily suited. In the case of forensic analysis, the primary goal is identification of individuals. Thus, it is important to obtain an STR profile that allows unambiguous identification of a suspect or an unknown person. Since identification is largely based on database searches, the choice of the appropriate STR markers is influenced by the fact that only selected STR systems are represented in large forensic databases like the Combined DNA Index System (CODIS). The STR systems included in these databases have been chosen on the basis of international agreements and standards, which are not necessarily based on maximum informativeness. In chimerism analysis, the starting point is substantially different. Although discrimination of individuals is obviously important as well, the requirements are different, since the individuals involved (donor and recipient) are known. Thus, an important selection criterion for an informative marker is not the difference per se, but the ability to identify even small amounts of residual cells in the mixture. In this regard, another critical aspect of STR analysis for chimerism analysis is the presence of additional signals (so-called stutter peaks). These artefacts are supposed to result from slipped-strand mispairing during amplification. The intensity of the stutter signals usually is about 2-10% of the corresponding STR allele. A high rate of stutter peaks has been reported for long simple repeat runs, whereas the presence of imperfect repeats and the use of DNA polymerases with increased processivity reduces the formation of such peaks. If stutter signals are present and coelute with the corresponding STR alleles of the recipient or the donor, they hamper accurate quantification. This is particularly important in the situation of low residual host cell levels and makes detection of minimal residual chimerism (reflecting e.g. residual disease) virtually impossible. Thus an optimal STR system for chimerism analysis has to have distinct signals for donor and recipient, which are not influenced by the stutter signals and other features affecting quantitative analysis of chimerism (Watzinger et al., Leukemia 2006). Taken together, the demands in the field of chimerism analysis and forensic diagnostics show obvious and important differences, which need to be addressed in order to optimize chimerism testing.

The marker panel of the present invention has also been evaluated in comparison to a commercially available multiplex microsatellite kit for forensic purposes (PowerPlex16; Promega; Penta E, D18S51, D21S11, TH01, D3S1358, FGA, TPOX, D8S1179, vWA, Amelogenin, Penta D, CSF1PO, D16S539, D7S820, D13S317 and D5S818). This kit has a marker composition, which is very similar to other commercial products marketed by Promega and other companies. Based on the results obtained, the panel of markers according to the present invention turned out to be much superior to the commercial kit in terms of informativeness by the stringent requirements of chimerism analysis (see examples).

Another aspect of the present invention relates to the use of a method according to the present invention for the detection or quantitative analysis of chimerism in a transplant recipient employing one or more markers (i.e. by performing singleplex or multiplex reactions).

Yet another aspect of the present invention relates to the use of a method according to the present invention for evaluating the risks of rejection or relapse of an individual subjected to transplantation.

The method according to the present invention may also be used to evaluate the risks of a graft rejection or disease relapse in a recipient who was subjected to transplantation, in particular to bone marrow or hematopoietic stem cell transplantation. In particular, said method allows to monitor the alleles of the leukocytes or other nucleated cells in said individual prior and after transplantation and to monitor the allelic profile of the recipient in respect to the profile of the donor over the time. A rejection or relapse can be detected when the allelic profile of specific recipient cells after transplantation reverses to the profile before transplantation.

Another aspect of the present invention relates to the use of a method according to the present invention for monitoring successful engraftment or the progress of healing of an individual subjected to transplantation.

Paternity testing can also be performed by determining the allelic profile of a first individual and by comparing said profile to a profile of a second individual suspected to be related to the first individual.

Another aspect of the present invention relates to the use of a method according to the present invention for paternity testing.

The method according to the present invention is also suited for paternity testing.

Another aspect of the present invention relates to the use of a method according to the present invention for genetic fingerprinting.

The method according to the present invention may be useful for establishing an allelic profile (a genetic fingerprint) of an individual. The genetic fingerprint obtained may be used for comparative analysis like forensic analysis or for tracing the origin of human specimens of unknown or uncertain source.

The present invention is further illustrated by the following examples and figures without being restricted thereto.

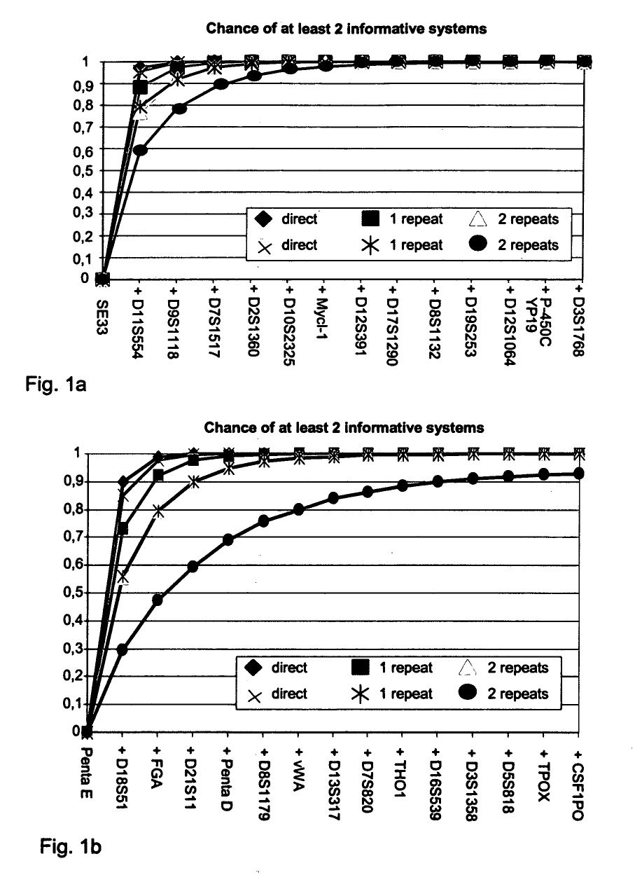

FIGS. 1a and 1b show a comparison of the level of informativeness of the entire set of chimerism markers (13 markers) according to the present invention (FIG. 1a) and PowerPlex 16 kit (FIG. 1b; Promega US; allows the co-amplification of Penta E, D18S51, D21S11, TH01, D3S1358, FGA, TPOX, D8S1179, vWA, Amelogenin, Penta D, CSF1PO, D16S539, D7S820, D13S317 and D5S818). Overall, the marker panel of the present invention can be expected to provide at least one marker adequate for quantitative chimerism analysis in >99% and two or more adequate markers in 91% of related donor/recipient pairs. This level of informativeness is not achievable with the PowerPlex 16 kit (FIG. 1b).

FIG. 2 shows the capacity of the marker panel to determine quantitative differences between serial clinical samples. It is shown that the intrinsic variability of the assay is only +2% in the great majority of instances.

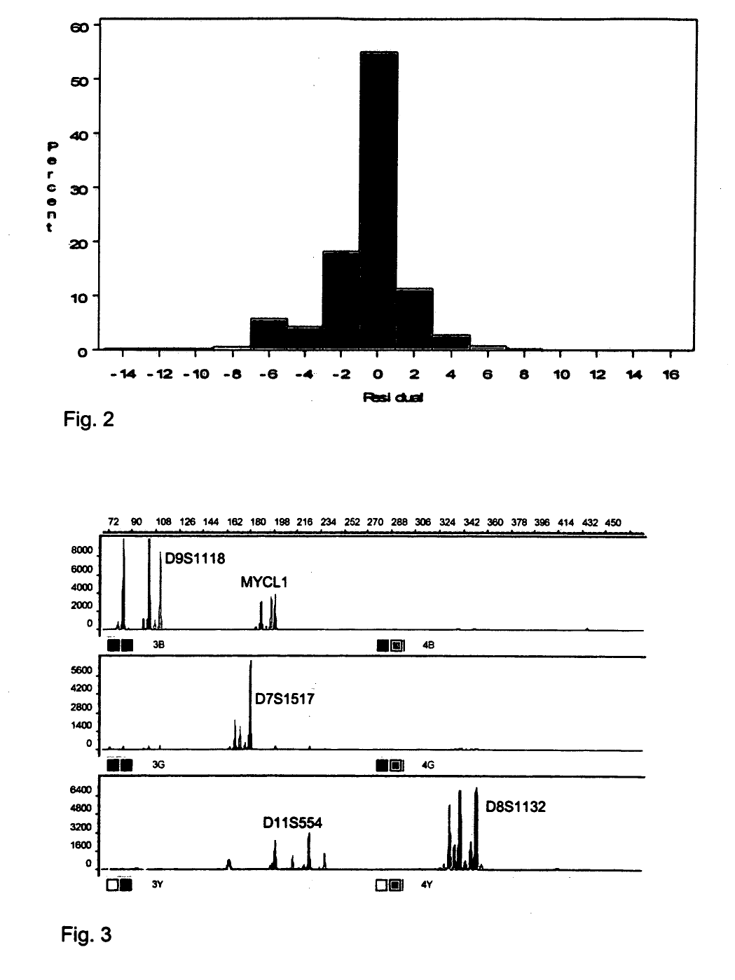

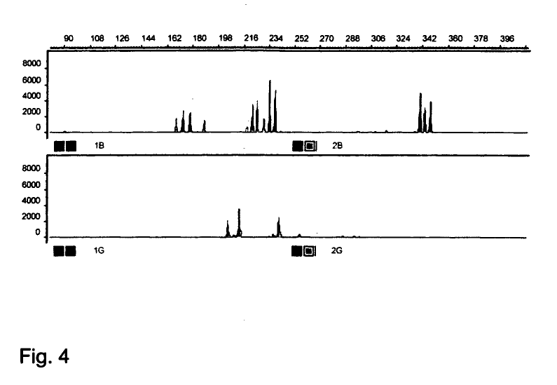

FIG. 3 shows an example of recipient and donor genotyping using the Multiplex group 1. The upper lane shows the markers D9S1118, MYCL1 (recipient heterozygous, donor homozygous) the middle lane D7S1517 (recipient homozygous, donor heterozygous), and the bottom lane D11S554 (donor and recipient heterozygous) and D8S1132 (donor and recipient heterozygous, one allele is shared).

FIG. 4 shows an example of recipient and donor genotyping using the Multiplex group 1. The upper lane shows the markers D10S2325 (donor and recipient heterozygous), D12S391 (donor and recipient heterozygous), and P450CYP19 (recipient heterozygous, donor homozygous), and the bottom lane D2S1360 (recipient heterozygous, donor homozygous).

### EXAMPLES

### Example 1

### Microsatellite Marker Panel and Conditions of PCR Amplification

The panel of microsatellite markers, the sequences of forward and reverse primers, the fluorescence label attached to the 5′ end of each forward primer, and the range of possible PCR products are indicated in Table 1.

TABLE 1

| | | | |
|---|---|---|---|
| | | Length of PCR | SEQ ID |
| Marker | Label | products | No. |

| | | | | | |
|---|---|---|---|---|---|
| D2S1360 | FW | CTGCATTAAAACATTCGAAACCAA | JOE | 233-273 | 1 |
| | REV | GCAGCAGATTGTGGGACTTCTCA | | | 2 |
| D7S1517 | FW | AGCCTGATCATTACCAGGT | JOE | 164-212 | 3 |
| | REV | GTTTCTATTGGGGCCATCTTGC | | | 4 |
| D8S1132 | FW | TCTCTCTCTCCCTCTCTCTTTCGAG | TMR | 347-379 | 5 |
| | REV | GTTTGCCATCTTCTTACCTCTGTTGGTC | | | 6 |
| D9S1118 | FW | CAGGATATTATGTGATGGAATCC | FL | 92-128 | 7 |
| | REV | GATCTCTTCTCTCTCTCTCTTTCTCCC | | | 8 |
| D10S2325 | FW | TATGGTGACCTTAAGCAGCCATG | FL | 163-213 | 9 |
| | REV | GTGTCTTAGCTGAGAGATCACGCACTGC | | | 10 |
| D11S554 | FW | GGTAGCAGAGCAAGACTGTC | TMR | 166-246 | 11 |
| | REV | GTTTCACCTTCATCCTAAGGCAGC | | 176-335 (GEN) | 12 |
| D12S1064 | FW | ACTACTCCAAGGTTCCAGCC | TMR | 114-142 | 13 |
| | REV | ACTGTTATCTCTCTTGTGGTAG | | | 14 |
| D12S391 | FW | ATCAACAGGATCAATGGATGCAT | FL | 237-269 | 15 |
| | REV | GGGCTTTTAGACCTGGACTGAG | | | 16 |
| D17S1290 | FW | CCAACAGAGCAAGACTGTC | FL | 169-205 | 17 |
| | REV | GTTTGAAACAGTTAAATGGCCAAAG | | | 18 |
| D19S253 | FW | ATAGACAGACAGACGGACTG | TMR | 239-274 | 19 |
| | REV | GTTTGGGAGTGGAGATTACCCCT | | | 20 |
| MYCL1 | FW | AACCGTAGCCTGGCGAGACT | FL | 156-225 | 21 |
| | REV | GTTTCCTTTTAAGCTGCAACAATTTC | | | 22 |
| P450CYP19 | FW | GTTCCACATAATGAAGCACAATC | FL | 314-464 | 23 |
| | REV | GTTTAATCGCCTGAGTCCTGGGA | | | 24 |
| SE-33 | FW | AATCTGGGCGACAAGAGTGA | TMR | 138-300 | 25 |
| | REV | ACATCTCCCCTACCGCTATA | | | 26 |

In Tables 2A and 2B the PCR reaction set-up is outlined.

TABLE 2A
Set-up for 1 PCR reaction (Primer concentrations)

| STR marker | Primer conc.* (pmol/μl) |
|---|---|
| D2S1360 | 2 |
| D7S1517 | 4 |
| D8S1132 | 2 |
| D9S1118 | * |
| D10S2325 | 2 |
| D11S554 | 2 |
| D12S1064 | 4 |
| D12S391 | 4 |
| D17S1290 | 2 |
| D19S253 | 4 |
| MYCL1 | 4 |
| P450CYP19 | 6 |
| SE33 | 4 |

* D9S1118 asymmetric PCR: Fw primer 8 pmol/ul and Rev primer 2 pmol/μl

TABLE 2B
Set-up for 1 PCR reaction (Master-Mix)

| | |
|---|---|
| PCR buffer II | 2.5 μl |
| (without MgCl₂) | |
| dNTP's (10 mM) | 1 μl |
| MgCl₂ (25 mM) | 2 μl |
| 5′-primer (2-8 pmol/μl)* | 1 μl |
| 3′-primer (2-6 pmol/μl)* | 1 μl |
| Taq Gold (5 U/μl) | 0.2 μl |
| H₂0 | 16.3 μl |
| DNA (10 ng) | 1 μl |
| Total volume | 25 μl |

The following cycling conditions (GeneAmp PCR System 9600 Thermal Cycler) were applied:

| | |
|---|---|
| 95° C. 11 min | |
| 96° C. 1 min | |
| ramp 100% to 94° C. for 30 sec | 10 cycles |
| ramp 29% to 60° C. for 30 sec | |
| ramp 23% to 70° C. for 45 sec | |
| ramp 100% to 90° C. for 30 sec | 20 cycles |
| ramp 29% to 60° C. for 30 sec | |
| ramp 23% to 70° C. for 45 sec | |
| 60° C. for 30 min | |
| 4° C. soak | |

### Example 2

### PCR Product Analysis by Capillary Electrophoresis and Fluorescence-Based Detection

1 μl PCR product was mixed with 24 μl HiDi-Formamide (Applied Biosystems) and 1 μl of the ILS 600 length standard (Promega). After denaturation at 95° C. for 3 min, the solution was loaded onto the capillary electrophoresis instrument (e.g. ABI3100). Following installation of the appropriate matrix, the analysis was performed using standard conditions. The injection parameters (voltage and injection time) were adjusted to achieve peak heights ranging around or above 5000 rfu.

### Example 3

### Limit of Detection and Quantitative Assessment of Patient-Donor Chimerism

The sensitivity of individual markers from the marker panel of the present invention (see example 1) to detect small numbers of recipient cells against a background of donor cells has been tested as outlined below. Moreover, the ability of individual markers to reveal changes in the proportion of recipient cells and to assess their adequacy for quantitative analysis of chimerism has been extensively tested.

To determine the performance of the markers in quantitative monitoring of chimerism, serial dilutions containing different proportions of recipient cell material were analyzed. The ability of each marker system to detect changes in the donor/recipient cell ratio has been determined. Robustness and reproducibility of quantitative analysis and inter-laboratory variation of the test under uniform experimental conditions have been assessed. Serial dilutions of recipient in donor DNA were centrally prepared and tested by individual laboratories.

The maximum sensitivity (i.e. limit of detection) was mostly at or below 1.6% (Table 3). The samples containing 1 and 10 ng of DNA template displayed similar sensitivity, but the robustness of the assay, as revealed by the reproducibility of individual PCR reactions, was better with the larger template amount. The observed limits of detection were satisfactory from the perspective of clinical application and the panel was shown to provide a robust system for quantitative chimerism analysis.

TABLE 3
Chimerism markers and detection limits

Detection Limit

Frequency

| Marker | 0.78 | 1.56 | 3.125 | 6.25 | Total |
|---|---|---|---|---|---|
| 1 | 11 | 4 | 1 | 0 | 16 |
| 2 | 11 | 3 | 0 | 0 | 14 |
| 3 | 11 | 2 | 3 | 0 | 16 |
| 4 | 4 | 9 | 2 | 1 | 16 |
| 5 | 10 | 5 | 1 | 0 | 16 |
| 6 | 15 | 7 | 2 | 0 | 24 |
| 7 | 21 | 2 | 1 | 0 | 24 |
| 8 | 9 | 4 | 3 | 0 | 16 |
| 9 | 8 | 0 | 0 | 0 | 8 |
| 10 | 16 | 0 | 0 | 0 | 16 |
| 11 | 6 | 7 | 7 | 2 | 22 |
| 12 | 7 | 6 | 3 | 0 | 16 |
| 13 | 16 | 0 | 0 | 0 | 16 |
| Total | 145 | 49 | 23 | 3 | 220 |

As shown in Table 3, the detection limit (DL) for the 13 markers of the marker panel was between 0.8-1.6% in 88% of the samples analyzed.

### Example 4

### Dynamic Range and Intrinsic Variability of the Assay

The dynamic range of the marker panel for quantitative assessment of chimerism is between 1-100%. The precision in determining the quantity of the subdominant cell population is higher within the range between 10-100% donor- or recipient-derived cells, while there is a tendency to overestimate the percentage of the subdominant cell population within the range between 1-10%. The capacity of the marker panel to determine quantitative differences between serial clinical samples is illustrated in FIG. 2, which reveals that the intrinsic variability of the assay is only +2% in the great majority of instances.

### Example 5

### Multiplex Reactions

Multiplex assays permitting co-amplification of different chimerism markers in the same PCR reaction have been established to facilitate patient/donor genotyping. The composition of the established multiplex reactions and the probability of identifying a minimum of two informative STR markers in a given patient/donor setting (one- and two-directional; one or two repeat units apart) are indicated in Tables 4 and 5. Examples of genotyping by multiplex group 1 and 2 are illustrated in FIGS. 3 and 4.

TABLE 4
STR-marker composition of multiplex reactions

| Multiplex 1 | Multiplex 2 |
|---|---|
| D11S554 | P-450CYP19 |
| D8S1132 | D12S391 |
| D9S1118 | D2S1360 |
| Mycl-1 | D10S2325 |
| D7S1517 | |

TABLE 5
The dyes used for 5′-labelling of the respective
forward primers are indicated. The range of allele sizes (in
base pairs) occurring in the population are shown. There is no
overlap of allele positions between markers labelled by the same
dye, thus permitting clear assignment of recipient and donor alleles
in the DNA specimens tested to individual markers.

| | FL (blue) | JOE (green) | TMR (yellow) |
|---|---|---|---|
| Group 1 | | | |
| D9S1118 | 92-128 | | |
| MYCL1 | 156-225 | | |
| D7S1517 | | 164-212 | |
| D11S554 | | | 166-246 |
| D8S1132 | | | 347-379 |
| Group 2 | | | |
| D10S2325 | | | 163-213 |
| D12S391 | 220-269 | | |
| P450CYP19 | 304-454 | | |
| D2S1360 | | 213-253 | |

### Example 6

### PCR Reaction Set-Up of the Multiplex Assays

The PCR reactions are preferentially set up in a total volume of 50 μl containing 47 μl premix (see Table 6), 2 μl polymerase (preferentially TaqGold) and 1 μl DNA (preferential concentration: 10 ng/μl). The amplification is carried out using the same PCR profile as for the singleplex reactions.

TABLE 6

| Premix for 1 reaction for | | Premix for 1 reaction | |
|---|---|---|---|
| group 1 contains: | | for group 2 contains: | |
| 10x PCR-buffer (ABI) | 5.0 μl | PE-buffer | 5.0 μl |
| dNTP's (10 mM) | 4.0 μl | dNTP's (10 mM) | 4.0 μl |
| MgCl2 (25 mM) | 4.0 μl | MgCl2 (25 mM) | 4.0 μl |
| 5′-D9S1118 (32 pmol/μl) | 1.0 μl | 5′-D10S2325 (18 pmol/μl) | 1.0 μl |
| 5′-D9S1118 (8 pmol/μl) | 1.0 μl | 3′-D10S2325 (18 pmol/μl) | 1.0 μl |
| 5′-MYCL1 (12 pmol/μl) | 1.0 μl | 5′-D12S391 (24 pmol/μl) | 1.0 μl |
| 3′-MYCL1 (12 pmol/μl) | 1.0 μl | 3′-D12S391 (24 pmol/μl) | 1.0 μl |
| 5′-D7S1517 (10 pmol/μl) | 1.0 μl | 5′-P450CYP19 (40 pmol/μl) | 1.0 μl |
| 3′-D7S1517 (10 pmol/μl) | 1.0 μl | 3′-P450CYP19 (40 pmol/μl) | 1.0 μl |
| 5′-D11S554 (20 pmol/μl) | 1.0 μl | 5′-D2S1360 (40 pmol/μl) | 1.0 μl |
| 3′-D11S554 (20 pmol/μl) | 1.0 μl | 3′-D2S1360 (40 pmol/μl) | 1.0 μl |
| 5′-D8S1132 (20 pmol/μl) | 1.0 μl | | |
| 3′-D8S1132 (20 pmol/μl) | 1.0 μl | | |
| H₂0 | 24.0 μl | H₂0 | 26.0 μl |
| Total volume | 47.0 μl | Total volume | 47.0 μl |

The high level of informativeness of the EUC markers, i.e. the ability to provide one or more microsatellite markers eligible for clinical chimerism testing in any donor-recipient constellation, complying with the stringent criteria established by the Eurochimerism consortium (Watzinger et al., Leukemia 2006), is indicated in Table 7.

TABLE 7

| | 1-directional | | | 2-directional | | |
|---|---|---|---|---|---|---|
| | direct | 1 repeat | 2 repeats | direct | 1 repeat | 2 repeats |
| Multiplex 1 | 0.999977 | 0.997232 | 0.979695 | 0.999874 | 0.976533 | 0.845469 |
| Multiplex 2 | 0.997202 | 0.967625 | 0.889619 | 0.990358 | 0.862584 | 0.575807 |
| Multiplex 1 + 2 | 1.000000 | 0.999992 | 0.999631 | 1.000000 | 0.999445 | 0.977175 |
| Multiplex 1 + 2 + | 1.000000 | 1.000000 | 0.999997 | 1.000000 | 0.999994 | 0.998392 |
| remaining markers | | | | | | |

1.-26. (canceled)

27. A method for simultaneously determining alleles present in a set of loci from at least one nucleic acid sample comprising:
a) providing the at least one nucleic acid sample;
b) subjecting the sample to nucleic acid amplification using primer pairs specific and optimized for each of the loci D2S1360, D7S1517, D8S1132, D9S1118, D10S2325, D11S554, D12S391, MYCL1 and P450CYP1; and
c) evaluating the length of amplification products obtained from step b) to determine the alleles present at each of the loci analyzed in the set within the sample.

28. The method of claim 27, wherein the sample is further subjected to nucleic acid amplification using primer pairs specific and optimized for at least one of the loci D12S1064, D17S1290, D19S253, and/or SE-33.

29. The method of claim 27, wherein the at least one nucleic acid sample is obtained from a transplantation recipient prior and after subjecting the recipient to a transplantation from a donor.

30. The method of claim 29, wherein at least one further nucleic acid sample is obtained from the donor.

31. The method of claim 29, wherein the recipient is transplanted with donor tissue.

32. The method of claim 31, wherein the donor tissue is bone marrow or hematopoietic stem cells.

33. The method of claim 29, wherein the determined lengths of the fragments obtained by step b) of the at least one sample of the recipient after transplantation are compared to the at least one sample of the recipient prior to transplantation or to the at least one sample of the donor.

34. The method of claim 29, wherein the relative quantity of donor and recipient derived cells in the sample is determined by quantifying the amplification products.

35. The method of claim 27, wherein the sample is a blood sample or a bone marrow sample.

36. The method of claim 27, wherein the sample comprises nucleated cells.

37. The method of claim 36, wherein the nucleated cells are leukocytes and/or stem cells.

38. The method of claim 37, wherein the nucleated cells are leukocytes further defined as granulocytes, monocytes, lymphocytes, B cells, T cells, or T suppressor cells.

39. The method of claim 38, wherein the leukocytes are further defined as NK cells, T helper cells, or cells expressing CD3, CD4, CD8, CD14, CD15, CD19, CD34, CD38, CD45 and/or CD56.

40. The method of claim 27, wherein the forward and/or the reverse primer are labelled.

41. The method of claim 40, wherein the forward and/or the reverse primer are labelled with a fluorescent marker.

42. The method of claim 41, wherein the fluorescent marker is Alexa 350, Alexa 430, FL, R6G, TMR, TRX, Cascade Blue, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, or Texas Red.

43. The method of claim 27, wherein the primer pairs comprise primer sequences SEQ ID NO: 1 and SEQ ID NO: 2 if a locus to be amplified is D2S1360; SEQ ID NO: 3 and SEQ ID NO: 4 if a locus to be amplified is D7S1517; SEQ ID NO: 5 and SEQ ID NO: 6 if a locus to be amplified is D8S1132; SEQ ID NO: 7 and SEQ ID NO: 8 if a locus to be amplified is D9S1118; SEQ ID NO: 9 and SEQ ID NO: 10 if a locus to be amplified is D10S2325; SEQ ID NO: 11 and SEQ ID NO: 12 if a locus to be amplified is D11S554; SEQ ID NO: 13 and SEQ ID NO: 14 if a locus to be amplified is D12S1064; SEQ ID NO: 15 and SEQ ID NO: 16 if a locus to be amplified is D12S391; SEQ ID NO: 17 and SEQ ID NO: 18 if a locus to be amplified is D17S1290; SEQ ID NO: 19 and SEQ ID NO: 20 if a locus to be amplified is D19S253; SEQ ID NO: 21 and SEQ ID NO: 22 if a locus to be amplified is MYCL1; SEQ ID NO: 23 and SEQ ID NO: 24 if a locus to be amplified is P450CYP19; and/or SEQ ID NO: 25 and SEQ ID NO: 26 if a locus to be amplified is SE-33.

44. The method of claim 27, wherein the nucleic acid of the nucleic acid sample is isolated from a nucleic acid comprising source.

45. The method of claim 27, wherein the lengths of the amplification products are evaluated by electrophoresis.

46. The method of claim 45, wherein the lengths of the amplification products are evaluated by gel or capillary electrophoresis.

47. The method of claim 27, wherein step b) is performed in more than one amplification reaction.

48. The method of claim 27, wherein the loci D11S554, D7S1517, D8S1132, D9S1118 and MYCL1 are simultaneously amplified in a first amplification reaction and the loci D2S1360, D10S2325, D12S391 and P450CYP19 are simultaneously amplified in a second amplification reaction.

49. The method of claim 27, further defined as a method of detection or quantitative analysis of chimerism in a transplant recipient employing one or more markers.

50. The method of claim 27, further defined as a method of evaluating the risks of rejection or relapse of an individual subjected to a transplantation.

51. The method of claim 27, further defined as a method of monitoring successful engraftment or the progress of healing of an individual subjected to a transplantation.

52. The method of claim 27, further defined as a method of paternity testing.

53. The method of claim 27, further defined as a method of genetic fingerprinting.

54. A kit for determining alleles present in a set of loci from at least one nucleic acid sample or for determining fragment lengths of alleles or for detecting chimerism in a transplant recipient comprising primer pairs each specific for at least three loci selected from the group consisting of D2S1360, D7S1517, D8S1132, D9S1118, D10S2325, D11S554, D12S391, MYCL1 and P450CYP19.

55. The kit of claim 54, further comprising primer pairs each specific for at least one of D12S1064, D17S1290, D19S253 and/or SE-33.

56. The kit of claim 54, wherein the forward and/or the reverse primer are labelled.

57. The kit of claim 57, wherein the forward and/or the reverse primer are labelled with a fluorescent marker.

58. The kit of claim 57, wherein the fluorescent marker is Alexa 350, Alexa 430, FL, R6G, TMR, TRX, Cascade Blue, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, or Texas Red.

59. The kit of claim 54, wherein the primer pairs have primer sequences SEQ ID NO: 1 and SEQ ID NO: 2 if a locus to be amplified is D2S1360; SEQ ID NO: 3 and SEQ ID NO: 4 if a locus to be amplified is D7S1517; SEQ ID NO: 5 and SEQ ID NO: 6 if a locus to be amplified is D8S1132; SEQ ID NO: 7 and SEQ ID NO: 8 if a locus to be amplified is D9S1118; SEQ ID NO: 9 and SEQ ID NO: 10 if a locus to be amplified is D10S2325; SEQ ID NO: 11 and SEQ ID NO: 12 if a locus to be amplified is D11S554; SEQ ID NO: 13 and SEQ ID NO: 14 if a locus to be amplified is D12S1064; SEQ ID NO: 15 and SEQ ID NO: 16 if a locus to be amplified is D12S391; SEQ ID NO: 17 and SEQ ID NO: 18 if a locus to be amplified is D17S1290; SEQ ID NO: 19 and SEQ ID NO: 20 if a locus to be amplified is D19S253; SEQ ID NO: 21 and SEQ ID NO: 22 if a locus to be amplified is MYCL1; SEQ ID NO: 23 and SEQ ID NO: 24 if a locus to be amplified is P450CYP19 and SEQ ID NO: 25 and SEQ ID NO: 26 if a locus to be amplified is SE-33.
